SEQUENCE LISTING (1 sequence)

SEQ ID NO: 1 - protein, 406 aa, Homo sapiens [sequence omitted]

(19) United States
(12) Patent Application Publication
(10) Pub. No.: US 2005/0069551 A1
(43) Pub. Date: Mar. 31, 2005

(54) Cytotoxic compound-protein conjugates as suppressors of tumor growth and angiogenesis

(75) Inventors: Mamoru Shoji, Conyers, GA (US); James P. Snyder, Atlanta, GA (US); Dennis C. Liotta, Atlanta, GA (US); Aiming Sun, Atlanta, GA (US)

Correspondence Address: PATREA L. PABST;PABST PATENT GROUP LLP, 400 COLONY SQUARE, SUITE 1200, ATLANTA, GA, 30361, US

(73) Assignee: Emory University

(21) Appl. No.: 10/900,490

(22) Filed: Jul. 28, 2004

Related U.S. Application Data
(63) Continuation-in-part of application No. 10/383,898, filed on Mar. 7, 2003.
(60) Provisional application No. 60/490,656, filed on Jul. 28, 2003.
(60) Provisional application No. 60/362,762, filed on Mar. 8, 2002.

(51) Int. Cl. A61K 39/395; C07K 16/46

(52) U.S. Cl. 424/178.1; 530/391.1

(57) ABSTRACT

Compositions and methods are provided for delivering cytotoxic compounds, such as natural curcumoids and synthetic curcumin analogs, specifically to cancer cells and to blood vessels that nourish solid tumors. The compositions include a cytotoxic drug tethered to a protein, such as factor VIIa, which can bind with high affinity to a receptor, such as tissue factor, expressed on the surface of cancer cells and vascular endothelial cells within the tumor microenvironment. Upon binding, the drug-protein-receptor complex is endocytosed and the drug is subsequently liberated inside the target cell via proteolytic cleavage. The compositions and methods may increase the efficacy of the cytotoxic agets and decrease their side effects by delivering the agents to specific target cells, such as cancer cells, vascular endothelial cells in a tumor, and metastatic foci anywhere in the body, providing the target cells express surface bound tissue factor. Additionally, methods of synthesis of cytotoxic compound-protein conjugates are provided, for example, curcuminoid-tether-linker-factor VIIa composition, as well as pharmaceutically acceptable compositions and methods for delivering a therapeutically-effective amount of a cytotoxic compound-protein conjugate together with one or more pharmaceutically acceptable carriers (additives) and/or diluents to an animal or human patient.

### CROSS-REFERENCE TO RELATED APPLICATIONS

This application claims priority to U.S. Provisional Application No. 60/490,656, entitled “Novel Curcuminoid-Factor VIIa Constructs as Suppressors of Tumor Growth and Angiogenesis, filed Jul. 28, 2003.

### STATEMENT REGARDING FEDERALLY SPONSORED RESEARCH OR DEVELOPMENT

This invention was made, at least in part, with funding from the National Institutes of Health with grant 1 R21 CA82995-01A1 and Department of Defense, Department of U.S. Army grant DAMD17-00-1-0241 awarded to Dr. Mamoru Shoji, Dr. Dennis Liotta, Dr. Jim Snyder and Dr. Aiming Sun. Accordingly, the United States Government has certain rights in this invention.

### BACKGROUND OF THE INVENTION

The present invention relates to novel compositions for selectively delivering a cytotoxic compound to a target cell. The present invention further relates to methods for synthesizing said novel compositions and for delivering them to tissue factor-bearing target cells.

The association between malignant disease and the hypercoagulable state was documented more than 100 years ago. A critical role for tumor-derived vasoactive factors like vascular endothelial growth factor (VEGF) in the formation of the blood vessels that nourish tumors has been emphasized in more recent work. Cell-associated procoagulants like tissue factor (TF) have also been implicated in the pathogenesis of these events. “Tissue factor” is a transmembrane protein specifically complexing with coagulation factor VII (and its activated form factor VIIa (fVIIa)), and is the primary regulator of blood coagulation. When bound to the extracellular domain of TF, fVIIa activates factor X (fX) via the extrinsic pathway. Alternatively, TF-VIIa indirectly activates fX via the activation of factor IX in the intrinsic blood clotting pathway.

Independent of the potent procoagulant function, TF may act as a modulator of VEGF expression and as a cell signal transducer. These studies have provided important evidence for a dynamic interaction between host inflammatory cells, tumor cells and vascular endothelial cells (VECs). “Leaky blood vessels,” perfusion of tumors with fibrinogen and conversion of the fibrinogen to fibrin by cell-associated procoagulants in the local tumor microenvironment are some of the consequences. These events may occur at the blood vessel wall during hematogenous spread of tumors or within the extravascular space as primary tumors or metastasis grow. Fibrin may be generated by the expression of procoagulant activity, particularly tissue factor expressed on the surface of tumor cells, tumor-associated macrophages and tumor-associated VECs.

Increased tumor angiogenesis is associated with a poor prognosis in a variety of human tumors, including invasive breast cancer, early stage and node negative breast cancer, prostate carcinoma and adenocarcinoma of the lung. There is a statistically significant correlation between so-called tumor microvascular density and relapse-free survival. It has been shown that tumor cells secrete a number of angiogenic factors, including VEGF, interleukin-8 (IL-8) and basic fibroblast growth factor (bFGF), and endothelial cell proliferation is faster in tumors compared with normal tissues.

Tumor cells secrete factors that increase vessel permeability. Vascular permeability factor, or VEGF, purified originally from tumor cells, has a molecular weight of 45 kDa and acts specifically on VECs to promote vascular permeability, endothelial cell growth and angiogenesis. VEGF induces expression of TF activity in VECs and monocytes and is chemotactic for monocytes, osteoblasts and VECs. VEGF promotes extravasation of plasma fibrinogen, which can be converted to fibrin by TF-dependent mechanisms. Fibrin deposition alters the tumor extracellular matrix to promote the migration of macrophages, fibroblasts and endothelial cells.

Overexpression of the TF gene in murine tumor cells leads to increased VEGF and decreased transcription of thrombospondin (TSP), an endogenous antiangiogenic factor. When grown in immunodeficient mice, the TF-producing cells stimulate angiogenesis by approximately 2-fold, whereas low TF producers inhibit angiogenesis. This effect of TF is independent of its clot-promoting activated procoagulation activity. Human melanoma cells, transfected to hyperexpress TF, demonstrate greater metastatic potential than those with low TF expression. This pro-metastatic effect of TF requires the procoagulant function of the extracellular domain of TF and its cytoplasmic domain. Tissue Factor, therefore, regulates angiogenic properties of tumor cells by regulating the production of growth regulatory molecules that can act on VECs. There is also a critical role for TF expression in blood vessel development in both mice and human embryos. TF appears to have the dual function of regulating angiogenesis and vasculogenesis.

Malignant human breast cancers and melanomas express high levels of TF and VEGF. TF is also expressed on the surface of vascular endothelial cells (VECs) within the tumor micro environment of invasive breast cancer and adenocarcinoma of the lung. There is a strong relationship between the synthesis of TF and VEGF levels in human breast cancer cell lines and in human melanoma cell lines, and there is co-localization of TF- and VEGF-specific mRNAs.

The signal for VEGF synthesis in cancer cells is mediated via serine residues of the TF cytoplasmic tail which contains two serine residues that can be substrates for protein kinase C. Expression of TF and VEGF in cancer cells is further enhanced under hypoxic condition, and TF may function as a growth factor receptor. Factor VIIa may induce cell signaling via PKC-dependent phosphorylation, mitogen-activated protein kinase (MAPK) pathways and subsequently, via the transcription factors NF-κB and AP-1. Curcumin (diferuloylmethane, 1,7-bis-(4-hydroxy-3-methoxyphenyl)-1,6-heptadiene-3,5-dione) is the major yellow pigment extracted from turmeric, the powdered rhizome of the herb Curcuma longa L. It is used as a spice to give a specific flavor and yellow color to curry. Turmeric has been a major component of the diet of the Indian subcontinent for several hundred years, and the average daily consumption of curcumin has been found to range to up to 0.6 grams for some individuals, without reported adverse effects. Turmeric has been discovered to be a rich source of phenolic compounds or curcuminoids. Curcuminoids refers to a group of phenolics present in turmeric, including curcumin, and other compounds chemically related to curcumin, such as demethoxycurcumin and bisdemethoxycurcumin. Curcumin has demonstrated both anticancer and anti-angiogenic properties. Its anti-tumor properties include growth inhibition and apoptosis induction in a variety of cancer cell lines in vitro, as well as the ability to inhibit tumorigenesis in vivo (Mehta et al. Anti-Cancer Drugs 8: 470-481 (1997); Kuo et al. Biochim and Biophys Acta 1317: 95-100 (1996); Jee et al. J. Invest Dermatol 111: 656-661 (1998); Kawamori et al. Cancer Res 59: 597-601 (1999); Singletary et al. Cancer Lett 103: 137-141 (1998); Ruby et al. Cancer Lett. 94: 79-83 (1995)). Curcumin's anti-angiogenesis effects include the inhibition of vascular endothelial cell (VEC) proliferation in vitro and capillary tube formation and growth in vivo (Arbiser et al., Arbiser et al., Molec. Med. 4:376-383 (1998); Thaloor et al., Cell Growth & Differentiation 9: 305-312 (1998)).

Curcumin also inhibits tumor necrosis factor- and phorbol ester-induced TF synthesis in VECs by blocking the transcription factors NF-κB, AP-1 and Egr-1. Curcumin can also inhibit TF and VEGF synthesis in human melanoma cell lines and prostate cancer cell lines, as well as bFGF-induced angiogenesis.

It is therefore an object of the invention to provide a method for inhibiting tumor growth, angiogenesis, and metastasis by delivering cytotoxic compounds, such as curcuminoids and synthetic curcumin derivatives, to a specific target, such as TF, which is aberrantly expressed on tumor cells and vascular endothelial cells in the tumor micro-environment.

It is another object to provide compositions that selectively target a surface marker, such as TF, on cancer cells and vascular endothelial cells within the tumor microenvironment, which contain a cytotoxic drug, such as a curcuminoid or synthetic curcumin derivative, tethered to a protein, such as factor VIIa, which can bind with high affinity to a surface marker.

It is yet another object of the invention to provide a method of coupling cytotoxic compounds, such as curcuminoids and synthetic curcumin derivatives, to proteins, such as factor VIIa and active-site inactivated factor VIIa, which can bind to cell surface markers, such as TF.

It is still another object of the invention to provide a method of coupling cytotoxic compounds, such as curcuminoids and synthetic curcumin derivatives, to proteins, such as factor VIIa and active-site inactivated factor VIIa, which maintains the affinity of the cytotoxic compound-protein conjugate for the cell surface marker.

It is yet another object of the invention to provide a method of increasing the solubility of factor VIIa conjugates in an aqueous solution.

### SUMMARY OF THE INVENTION

Compositions and methods are provided for delivering cytotoxic compounds, such as natural curcumoids and synthetic curcumin analogs, specifically to cancer cells and to blood vessels that nourish solid tumors. The compositions include a cytotoxic drug tethered to a protein, such as factor VIIa, which can bind with high affinity to a receptor, such as tissue factor, expressed on the surface of cancer cells and vascular endothelial cells within the tumor microenvironment. Upon binding, the drug-protein-receptor complex is endocytosed and the drug is subsequently liberated inside the target cell via proteolytic cleavage. The compositions and methods may increase the efficacy of the cytotoxic agets and decrease their side effects by delivering the agents to specific target cells, such as cancer cells, vascular endothelial cells in a tumor, and metastatic foci anywhere in the body, so long as the target cells express surface bound tissue factor. The compositions and methods are also useful for treating disease that requires targeted delivery of antiangiogenesis therapy including, but not limited to, cancer, reocclusion of the coronary artery, diabetic retinopathy, brain infarction, macular degeneration, and rheumatoid arthritis.

Additionally, methods of synthesis of cytotoxic compound-protein conjugates are provided, for example, curcuminoid-tether-linker-factor VIIa composition, as well as pharmaceutically acceptable compositions and methods for delivering a therapeutically-effective amount of a cytotoxic compound-protein conjugate together with one or more pharmaceutically acceptable carriers (additives) and/or diluents to an animal or human patient, and methods for enhancing the solubility in aqueous solution of the factor VIIa conjugates.

### BRIEF DESCRIPTION OF THE DRAWINGS

FIG. 1 is a graph of the the mean growth inhibitory concentrations of various curcuminoids when added to cultures of immortalized endothelial cells.

FIG. 2 is a graph of the mean growth inhibitory concentrations of various curcuminoids when tested against a panel of cultured tumor cells.

FIG. 3 is a graph of the mean growth inhibitory concentrations of various curcuminoids when added to cultures breast cancer cells.

### DETAILED DESCRIPTION OF THE INVENTION

I. Conjugate Components

The compositions comprise a drug covalently bonded via a linker to a protein capable of selectively binding to a cell surface maker and then being internalized. The compositions may also comprise a tether molecule that alone or in conjunction with the linker may serve to bond the cytotoxic compound to the protein.

In a preferred embodiment, the protein is factor VIIa, the cell surface marker is tissue factor, and the drug is a cytotoxic compound such as curcumin. Factor VIIa can bind with high affinity to tissue factor, which is aberrantly expressed on the surface of cancer cells and vascular endothelial cells in the tumor micro-environment. Upon binding, the drug-factor VIIa-tissue factor complex is endocytosed and the drug is subsequently liberated inside the target cell via proteolytic cleavage. Inhibition of tissue factor synthesis blocks vascular endothelial growth factor (VEGF) synthesis and tumor angiogenesis. The most preferred compound is the conjugate EF24-FFRck-fVIIa. As demonstrated in the Examples below, this construct kills cancer cell lines and vascular endothelial cells, such as HUVECs, that express tissue factor on the cell surface. The conjugate does not kill normal cells that do not express tissue factor. EF24-FFRck that is not coupled to fVIIa does not kill either cancer cells or normal cells regardless of the presence or absence of tissue factor expression on the cell surface because it cannot bind to any cells.

A. Cytotoxic Compounds

The term “cytotoxic compound” as used herein refers to a compound that, when delivered to a cell, either to the interior of a target cell or to the cell surface, is capable of killing the cell or otherwise inhibiting the proliferation of the target cell. The cytotoxic compound can be any such molecule that can form an amide or ester bond or otherwise be covalently bonded to a tether or a peptidyl linker and thereby connected to a protein that can selectively bind to a surface marker of a cell. In the preferred embodiment, the cytotoxic compound is a curcumin, which forms a stable conjugate with a linker coupling the curcumin to factor VII.

The term “angiogenesis inhibitor” as used herein refers to a compound or composition that, when administered as an effective dose to an animal or human, will inhibit or reduce the proliferation of vascular endothelial cells, thereby reducing the formation of neovascular capillaries. Angiogenesis inhibitors may be divided into at least two classes. The first class, direct angiogenesis inhibitors, includes those agents which are relatively specific for endothelial cells and have little effect on tumor cells. Examples of these include soluble vascular endothelial growth factor (VEGF) receptor antagonists and angiostatin. Indirect inhibitors may not have direct effects on endothelial cells but may down-regulate the production of an angiogenesis stimulator, such as VEGF. (Arbiser et al. (1998)). VEGF has been shown to be up-regulated during chemically induced skin carcinogenesis; this is likely due to activation of oncogenes such as H-ras. (Arbiser et al., Proc. Natl. Acad. Sci. U.S.A. 94:861-866 (1997)); (Larcher et al., Cancer Res. 56:5391-5396 (1996)); (Kohl et al., Nature Med. 1:792-797 (1995)). Examples of indirect inhibitors of angiogenesis include inhibitors of ras-mediated signal transduction, such as farnesyltransferase inhibitors. Direct inhibition of endothelial cell proliferation can be assayed in cell culture systems, in which the effects of specific factors which control the complex process of angiogenesis can be studied. Effects discovered in such in vitro systems can then be studied in in vivo systems as described, for example, by Kenyon et al., Invest. Ophthalmol. 37:1625-1632 (1996).

The term “curcumin (diferuloylmethane)” and certain of its analogs, together termed “curcuminoids,” as used herein, refers to a well known natural product, recognized as safe for ingestion by and administration to mammals including humans. (Bille et al., Food Chem. Toxicol. 23:967-971 (1985)). The term “curcuminoid” as used herein also refers to synthetic curcumin derivatives such as those disclosed in PCT Application Serial No. WO 01/40188. The fully saturated derivative tetrahydrocurcumin is included in the term curcuminoid. Curcumin can be obtained from many sources, including for example Sigma-Aldrich, Inc. The curcumin analogs demethoxycurcumin, bisdemethoxycurcumin and tetrahydrocurcumin can also be obtained from many sources, or readily prepared from curcumin by those skilled in the art.

The term “prodrug” is intended to encompass compounds which, under physiological conditions, may be converted into a pharmaceutically active curcuminoid. A common method for making a prodrug is to select moieties which are hydrolyzed under physiological conditions to provide the desired biologically active drug. In other embodiments, the prodrug may be converted by an enzymatic activity of the recipient animal or cell.

The term “aliphatic group” as used herein refers to a straight-chain, branched-chain, or cyclic aliphatic hydrocarbon group and includes saturated and unsaturated aliphatic groups, such as an alkyl group, an alkenyl group, and an alkynyl group.

The term “alkyl” as used herein refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, alkylaminos, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The term “aryl” refers to both substituted and unsubstituted aromatic rings. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocycle” refer to 4- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

The phrase “fused ring” is art recognized and refers to a cyclic moiety which can comprise from 4 to 8 atoms in its ring structure, and can also be substituted or unsubstituted, (e.g., cycloalkyl, a cycloalkenyl, an aryl, or a heterocyclic ring) that shares a pair of carbon atoms with another ring. To illustrate, the fused ring system can be a isobenzofuran and a isobenzofuranone.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines. The term “alkylamine” as used herein means an amine group, as defined above, having a substituted or unsubstituted alkyl attached thereto.

The term “amido” is art recognized as an amino-substituted carbonyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)m. Representative alkylthio groups include methylthio, ethylthio, and the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

Certain compounds may exist in particular geometric or stereoisomeric forms, including cis- and trans-isomers, R— and S-enantiomers, diastereomers, (D)-isomers, (L)-isomers, the racemic mixtures thereof, and other mixtures thereof. Additional asymmetric carbon atoms may be present in a substituent such as an alkyl group or other stereogenic centers. Likewise certain compounds can display overall molecular asymmetry without stereogenic centers leading to sterioisomers

Enantiomers may be prepared by asymmetric synthesis, or by derivitization with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts can be formed with an appropriate optically-active acid or base, followed by resolution of the diastereomers thus formed by fractional crystallization or chromatographic means well known in the art.

More than ninety novel curcumin analogs have been synthesized, as described in PCT Publication No. WO 01/40188. Several of these compounds suppress cancer cell VEGF production, but are not cytotoxic to either cancer cells or endothelial cells at concentrations where curcumin is otherwise cytotoxic. Any curcuminoid such as, but not limited to, those curcuminoids disclosed in PCT Publication No. WO 01/40188, may be used in the compositions if capable of bonding to a carboxylic or polycaproyl tether by reactions such as described, for example, in Example 2, below.

A particularly suitable curcuminoid is 3,5-Bis-(2-fluorobenzylidene)-piperidin-4-one (“EF24”) having the formula: 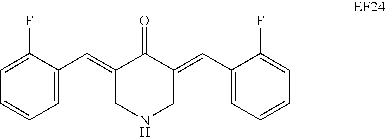
or a salt thereof.

Previous studies demonstrated that EF24 is about 10 times more potent than cisplatin, a well-known anticancer agent, against cancer cell lines in vitro (Adams et al. Biorg. Med. Chem. 12(14):3871-83 (2004)).

In various embodiments, the cytotoxic compound may be a curcuminoid having the formula:

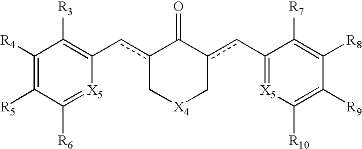

X₄ is (CH₂)m, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl; m is 1-7; each X₅ is independently N or C—R₁₁; and each R₃—R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂)ₘ, m is 2-6, and each X₅ is C—R₁₁, R₃—R₁₁ are not alkoxy, and when X₄ is NR₁₂ and each X₅ is N, R₃—R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

In some embodiments, X₄ is selected from the group consisting of —NH and —NR_{12,} and R₃—R₁₀ may be selected from hydroxyl and —NHR₁₂.

In one embodiment, the cytotoxic compound is a curcuminoid having the formula:

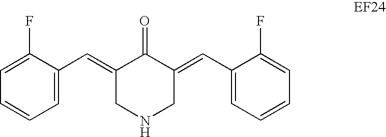

B. Polypeptide Targeting Cells that Endocytose Conjugate

Any polypeptide that can selectively bind to a cell surface marker such as, for example, an extracellular region of surface bound tissue factor and which, when so bound, may then be internalized by the targeted cell can be used.

As used herein the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptides” also contemplates polypeptides as defined above that are encoded by nucleic acids, produced through recombinant technology, isolated from an appropriate source, or are synthesized. The term “polypeptide” further contemplates polypeptides as defined above that include chemically modified amino acids or amino acids covalently or noncovalently linked to labeling ligands.

The term “truncated” as used herein refers to a polypeptide or protein that has fewer amino acids than a parent polypeptide or protein. It is contemplated that the difference in the amino acid sequence may be at one or both of the termini of an amino acid sequence or due to amino acids deleted from the interior of the sequence when compared to the parent amino acid sequence.

The terms “cell surface antigen” and “cell surface marker” as used herein may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” include, but are not limited to, CD5, CD30, CD34, CD45RO, CDw65, CD90 (Thy-1) antigen, CD117, CD38, and HLA-DR, AC133 defining a subset of CD34+ cells, CD19, CD20, CD24, CD10, CD13, CD33 and HLA-DR. Cell surface molecules include carbohydrates, proteins, lipoproteins or any other molecules or combinations thereof, that may be detected by selectively binding to a ligand or labeled molecule by methods such as, but not limited to, flow cytometry, FRIM, fluoresence microscopy and immunohistochemistry.

The term “tissue factor” as used herein refers to a transmembrane protein which complexes with coagulation factor VII (and the activated form factor VIIa (fVIIa)), and is the primary regulator of blood coagulation.

The term “fVII” means “single chain” coagulation factor VII that may have the amino acid sequence SEQ ID NO: 1, or a truncated or modified form thereof.

The term “factor VIIa”, or “fVIIa” means “two chain” activated coagulation factor VII cleaved by specific cleavage at the Arg152-Ile153 peptide bond. The uncleaved factor VII has the contiguous sequence as illustrated in SEQ ID NO: 1. Factor VIIa may be purified from blood or produced by recombinant means. The covalent bonding of the linker to the polypeptide may be to the uncleaved factor VII which is subsequently cleaved between the 152-153 amino acid positions, or to the cleaved fVIIa.

Human purified factor VIIa suitable is preferably made by DNA recombinant technology, e.g. as described by Hagen et al., Proc. Natl. Acad. Sci. USA 83: 2412-2416, (1986) or as described in European Patent No. 200.421. Factor VIIa produced by recombinant technology may be authentic factor VIIa or a more or less modified factor VIIa provided that such factor VIIa has substantially the same biological activity for blood coagulation as authentic factor VIIa. Such modified factor VIIa may be produced by modifying the nucleic acid sequence encoding factor VII either by altering the amino acid codons or by removal of some of the amino acid codons in the nucleic acid encoding the natural fVII by known means, e.g. by site-specific mutagenesis.

Factor VII may also be produced by the methods described by Broze & Majerus, J. Biol. Chem. 255 (4): 1242-1247, (1980) and Hedner & Kisiel, J. Clin. Invest. 71: 1836-1841, (1983). These methods yield factor VII without detectable amounts of other blood coagulation factors. An even further purified factor VII preparation may be obtained by including an additional gel filtration as the final purification step. Factor VII is then converted into activated fVIIa by known means, e.g. by several different plasma proteins, such as factor XIIa, IXa or Xa. Alternatively, as described by Bjoern et al. (Research Disclosure, 269 September 1986, pp. 564-565), factor VII may be activated by passing it through an ion-exchange chromatography column, such as Mono QR™ (Pharmacia Fine Chemicals).

The term “antibody” as used herein refers to polyclonal and monoclonal antibodies and fragments thereof, and immunologic binding equivalents thereof that are capable of selectively binding to a region of tissue factor. The term “antibody” refers to a homogeneous molecular entity, or a mixture such as a polyclonal serum product made up of a plurality of different molecular entities, and may further comprise any modified or derivatised variant thereof that retains the ability to specifically bind an epitope. A monoclonal antibody is capable of selectively binding to a target antigen or epitope.

Suitable polypeptides include, but are not limited to, factor VII or factor VIIa (fVIIa), tissue factor pathway inhibitor (TFPI) or an antibody capable of specifically binding to tissue factor. A preferred polypeptide is a component polypeptide of fVIIa derived from the amino acid sequence SEQ ID NO: 1, wherein before conjugation to the linker, the polypeptide may be the uncleaved SEQ ID NO: 1, or cleaved between amino acid positions 152-153 such that the component polypeptide receiving the linker may comprise the amino acid sequence between positions 1 and 152, 153-408 or derivatives thereof, of SEQ ID NO: 1.

The preferred polypeptide is fVIIa having at least 80% similarity to the amino acid sequence SEQ ID NO:1, cleaved between amino acid positions 152 and 153 or truncated derivatives or variants thereof. The fVIIa may be derived from any species, including human fVII. The fVIIa polypeptide may be truncated to include sequence variations by methods well known to those skilled in the art, including modification of cloned nucleic acid encoding all or part of SEQ ID NO:1, or by proteolytic cleavage of the fVIIa polypeptide, and the like. Any truncation or amino acid substitution will retain the ability of the modified fVIIa and or modified TFPI to selectively bind to tissue factor, be internalized by a target cell and capable of forming a covalent bond with a linker molecule having a chloromethylketone group thereon.

C. Linkers and Tethers

Linkers

The term “linker” as used herein refers a molecule capable of covalently connecting a cytotoxic compound to an amino acid side chain of a protein. The term “linker” may be a non-peptidyl linker or a peptidyl linker. The linker may have covalently bonded thereto a tether, as defined below, for covalently linking a cytotoxic compound to the linker. The term “peptidyl linker” as used herein refers to a peptide comprising at least two amino acids and which can be coupled to an amino acid side-chain of a protein. The linker may have a reactive group at the carboxyl terminus such as, but not limited to, a chloromethylketone. The peptide of the peptidyl linker may be cleavable by proteolytic enzymes found within a cell.

One linker suitable for use is a peptidyl methylketone linker covalently bonded to the polypeptide, most preferably to the side chain of an amino acid within the catalytic triad of the serine protease domain of fVIIa. In the human and bovine factor VII proteins, the amino acids which form a catalytic “triad” are Ser344, Asp242, and His193, numbering indicating position within the sequence SEQ ID NO:1. The catalytic sites in factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined by Sigler et al., J. Mol. Biol., 35:143-164 (1968), and determining from the alignment the analogous active site residues. Attachment of the peptidyl linker to this domain will inactivate the serine protease activity, thereby reducing the potential of the composition, when administered to an animal, to induce blood coagulation. In one preferred embodiment, at least one linker is covalently bonded to the His 198 position of SEQ ID NO: 1.

The terms “methylketone” and “chloromethylketone” as used herein refer to the carboxy terminus reactive moiety that may form the covalent bond between a peptide linker and an amino acid side chain of a recipient polypeptide. During the linkage reaction, the chloro group is removed. Thus, the unlinked peptidyl linker will have a chloromethylketone moiety and the covalently attached peptide will have a methylketone moiety without a halogen atom thereon.

Peptidyl linkers suitable for use, before being bonded to the polypeptide, have a carboxy-terminus chloromethylketone group that may react with a suitable amino acid side chain of the polypeptide, as described in Example 1, below. Preferably, but not necessarily, the carboxy terminal amino acid having the chloromethylketone group thereon is an arginine. Although any peptidyl chain sequence or length may be used, a suitable peptide is a tripeptide. Preferred peptidyl linkers include, but are not limited to, tyrosine-glycine-arginine-chloromethylketone (YGR-ck); phenylalanine-phenylalanine-arginine-chloromethylketone (FFR-ck), glutamine-glycine-arginine-chloromethylketone (QGR-ck), glutamate-glcine-arginine chloromethylketone (EGR-ck) and the like. A most preferred linker is FFR-ck. The stoichiometry of attachment of the curcuminoid EF24-tether-FFRck to fVIIa is given in Example 2, below.

It will be understood by those of skill in the art that upon covalently attaching the chloromethylketone to the recipient polypeptide, the chloro-moiety is displaced. Accordingly, the term “FFR-ck-VIIa”, for example, refers to FFR-methylketone tripeptidyl linker bonded to fVIIa and not having a chloro- atom attached thereto.

It is believed that a complex, formed from phenylalanyl-phenylalanyl-arginyl-ck-VIIa (FFR-ck-VIIa) and tissue factor (TF) expressed on the plasma membrane of cancer cells, may be internalized in a FFR-ck-VIIa concentration-dependent manner by ligand-receptor mediated endocytosis. The ligand-receptor complex is endocytosed into early and late endosomes and is delivered to lysosomal vesicles and degraded by lysosomal enzymes. Accordingly, the peptide selected for use as a linker peptide is also suitable for cleavage by an intracellular hydrolytic activity of the target cell enzyme. When so cleaved, after endocytotic internalization, the curcuminoid attached to the linker may be released from a polypeptide such as fVIIa. The released curcuminoid may then modulate a physiological function of the target cell.

A number of different linkers can be used. For example, linkers can be an arginyl methylketone such as phenylalanine-phenylalanine-arginine methylketone, tyrosine-glycine-arginine methylketone, glutamine-glycine-arginine methylketone, glutamate-glycine-arginine methylketone or phenylalanine-proline-arginine methylketone. In a preferred embodiment, the linker is phenylalanine-phenylalanine-arginine methylketone. In another preferred embodiment, the linker is tyrosine-glycine-arginine methylketone.

In another preferred embodiment, a linker is covalently bonded to an amino acid side chain within a serine protease active site of factor VIIa, thereby inactivating the serine protease active site.

Tethers

The term “tether” as used herein refers to a molecule that can form a hydrolysable bond such as, but not limited to, a carbamate, an amide, an ester, a carbonate or a sulfonate bond with a cytotoxic compound such as, but not limited to, a curcuminoid, and which can also be covalently bonded to a linker such as, but not limited to, the N-terminus of a linker, including a peptidyl linker, thereby connecting the cytotoxic compound to the linker via the tether. Suitable tethers include a dicarboxylic acid, a disulfonic acid, an omega-amino carboxylic acid, an omega-amino sulfonic acid, an omega-amino carboxysulfonic acid, or a derivative thereof, wherein the tether may comprise 2-6 carbons in any arrangement such as a linear, branched or cyclic carbon arrangement, and wherein the tether is capable of forming a hydrolysable bond.

The curcuminoid may be covalently bonded to a tether, which preferably is a dicarboxylic acid or caproyl moiety. Another exemplary tether is succinate that may be bonded to a curcuminoid by the addition of succinic anhydride, as described in Example 2, below. The hydrolysable bond can be a carbamate, an amide, an ester, a carbonate and a sulfonate.

In one embodiment, the cytotoxic compound is linked to the peptide via a tether that functions as a linker and as a tether. In a preferred embodiment, the cytotoxic compound is connected to the peptide via both a tether and a linker. The tether forms a hydrolysable bond with the cytotoxic compound and is covalently bonded to a linker, which is covalently bonded to an amino acid side chain of the protein.

In yet another preferred embodiment, the cytotoxic compound is bonded to a tether, which is covalently linked to an N-terminal amino acid of a peptidyl linker such as phenylalanine-phenylalanine-arginine, the C-terminal amino acid of which comprises a methylketone. The methylketone group forms a covalent bond with an amino acid side group of factor VIIa (fVIIa) that does not prevent the conjugated construct from selectively binding to tissue factor expressed on a cell membrane. Preferably, the curcuminoid-tether-linker will have bonded to an amino acid of the serine protease domain of the fVIIa, thereby blocking the procoagulating activity.

In yet other embodiments, the tether can be a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond. The tether can also be succinate.

II. Conjugates and Methods of Production

A curcuminoid-polypeptide conjugate can be prepared by
(a) synthesizing a product comprising a curcuminoid having a tether covalently bonded thereto, wherein the curcuminoid has the formula: 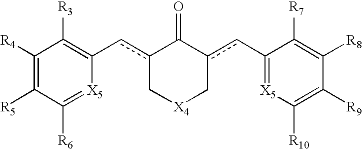
   wherein X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl, m is 1-7, each X₅ is independently N or C—R₁₁, and each R₃—R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, or trialkylammonium; and the dashed lines indicate optional double bonds; with the proviso that when X₄ is (CH₂)ₘ, m is 2-6, and each X₅ is C—R₁₁, R₃—R₁₁ are not alkoxy, and when X₄ is NR₁₂ and each X₅ is N, R₃—R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers,
(b) providing a peptidyl chloromethylketone linker,
(c) bonding covalently the product of step (a) and the linker, and
(d) covalently bonding the composition of step (c) to a polypeptide capable of selectively binding to tissue factor on the surface of a target cell.

In one embodiment, the method comprises the steps of synthesizing a product comprising a cytotoxic compound, bonding covalently the product of step (a) and the linker, and covalently bonding at least one molecule of the composition of step (b) to a protein capable of selectively binding to a surface marker of a target cell.

In one embodiment of this aspect, step (a) comprises reacting the curcuminoid with a tether such as a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

In various embodiments, X₄ is selected from the group consisting of —NH and —NR₁₂, and R₃—R₁₀ can be selected from hydroxyl and —NHR₁₂.

In a preferred embodiment, the cytotoxic compound has the formula:

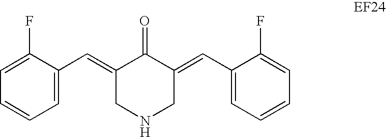

In another preferred embodiment, step (a) comprises reacting the cytotoxic compound with a dicarboxylic anhydride. In yet another preferred embodiment, the dicarboxylic anhydride is succinic anhydride.

In one embodiment, the product of step (a) has the formula: 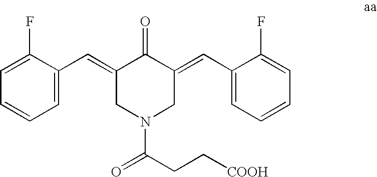
and in yet another embodiment, step (b) comprises providing a peptidyl linker.

In various embodiments, step (b) comprises the steps of reacting a composition having the formula: 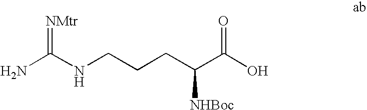
with isopropyl chloroformate and ethereal diazomethane, thereby producing a compound having the formula: 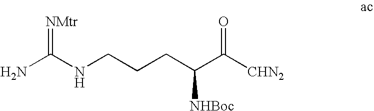
reacting a compound having the formula: 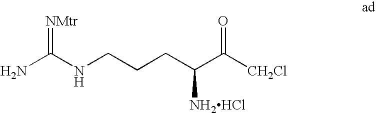
with N-Boc-Phe-Phe-OH, isopropyl chloroformate, and a base; thereby producing a compound having the formula: 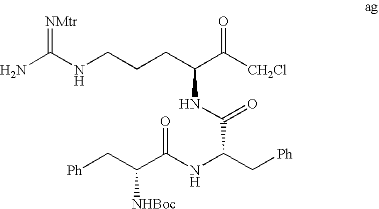
deprotecting compound ag, thereby producing a compound having the formula:

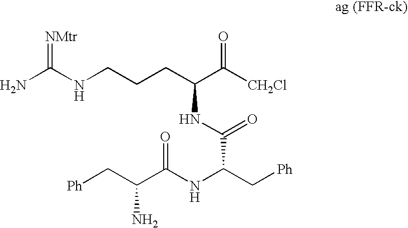

In one embodiment, the composition of step (b) has the formula: 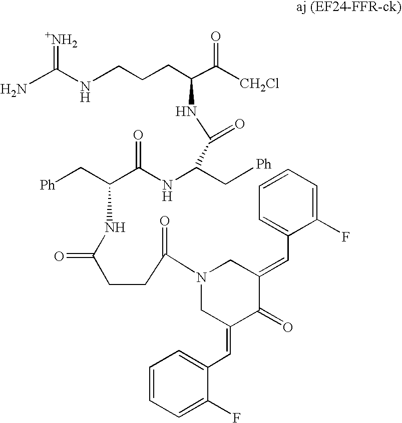
In a preferred embodiment, the protein is a component polypeptide of a factor VIIa.

In another embodiment at least one molecule of the composition of step (b) is covalently bonded to an amino acid of the serine protease active site of factor VIIa, thereby inactivating the active site.

In yet another embodiment, the amino acid is the His 193 of SEQ ID NO: 1.

Removal of Solvent and Solubilization

In a preferred embodiment, a solution of the protein is dialyzed to remove precipitate prior to the addition of the cytotoxic compound tethered to the linker. The solution may be dialyzed in any suitable buffer, such as n-(2-Acetamido)]-2-Aminoethanesulfonic Acid (ACES), tris[hydroxymethyl]aminomethane (Tris) (including Tris acetate, Tris acid, and Tris base), (3-cyclohexylamino)-1-propanesulfonic acid (CAPS), (3-[(3-cholamidopropyl) dimethylammonio]-1-propanesulfonate) (CHAPS), N-[2-hydroxyethyl]piperazine-N′-[2-ethanesulfonic acid] (HEPES), 2-(n-morpholino)ethanesulfonic acid (MES), 3-(N-Morpholino) Propanesulfonic Acid (MOPS), piperazine-n,n′-bis (2-ethanesulfonic acid), 1,4-piperazine diethanesulfonic acid (PIPES).

In a preferred embodiment, the buffer is Tris-Cl.

In another embodiment, the solution is dialyzed in a buffer containing a surfactant. Examples of suitable surfactants are Tween 20, Tween 80, or equivalents thereof.

In a another preferred embodiment, the cytotoxic compound tethered to the linker is dissolved in a solvent to provide a molar ratio of cytotoxic compound-tether-linker to protein of greater than 1 to 1, preferably 5 to 1, 10 to 1, and most preferably, 20 to 1.

The cytotoxic compound tethered to the linker may be dissolved in any pharmaceutically acceptable solvent including, nonaqueous, polar solvents such as oils, alcohols, amides, esters, ethers, ketones, hydrocarbons and mixtures thereof, as well as aqueous, pharmaceutically acceptable liquids, including water, saline solutions, dextrose solutions, electrolyte solutions, etc.

Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)-lactamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methyl pyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate®. 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄ -C₂₂ fatty acid(s)(e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.)(Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.,)(Marcel Dekker, Inc., New York, N.Y., 1980), Remington's Pharmaceutical Sciences (A. Gennaro, ed., 19th ed.)(Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, J. Pharm. Sci. 52(10): 917-927 (1963).

In a preferred embodiment, the solvent is dimethyl sulfoxide.

In another preferred embodiment, aliquots of a fraction of the volume of the compound-linker solution is added to a protein solution to avoid precipitation.

The cytotoxic compound-linker-protein conjugate mixture may be dialyzed to remove solvent and unconjugated free compound-linker. Suitable dialysis buffers are described above.

The solution of compound-linker-protein conjugate containing the concentrate may be concentrated to further filter out free compound-linker in addition to concentrate the conjugate solution.

II. Methods of Treatment

The phrase “therapeutically-effective amount” as used herein means that amount of a compound, material, or composition which is effective for producing a desired therapeutic effect against cancer or other pathological comprising neovascularization.

A. Pharmaceutically Acceptable Formulations

Pharmaceutically acceptable compositions are provided that comprise a therapeutically-effective amount of conjugate, formulated together with one or more pharmaceutically acceptable carriers (additives) and/or diluents for use as a therapeutic agent for the treatment of a pathological condition of an animal or human such as a cancer or a neovascular based disease.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or an encapsulating material such as liposomes, polyethylene glycol (PEG), PEGylated liposomes, or particles, which is compatible with the other ingredients of the formulation and not injurious to the patient. Examples of pharmaceutically acceptable excipients include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The compositions may be delivered to an animal or human by any of these routes, depending on the disorder to be treated. The preferred route of administration is intravenous injection so that the effective dose of the compound can be delivered to a tumor via the vascular system. The dose may be delivered by subcutaneous injection, intraperitoneal injection, direct injection into the tumor or a proximal blood vessel feeding the tumor for reducing dilution of the effective therapeutic composition, and to achieve more rapid application of the composition to the tissue factor-bearing target tumor and/or vascular cells. The affinity of the carrier polypeptide (i.e. fVIIa) for the cell surface marker (i.e. tissue factor) will localize the effective dose of the therapeutic composition for selectively targeting of proliferating tumor and endothelial cells contributing to neovascularization of a tumor and to prevent metastasis of the tumor cells themselves.

As described in detail below, the pharmaceutical compositions may be specially formulated for administration in solid or liquid form, including those adapted for oral administration or parenteral administration, for example, by subcutaneous, intramuscular or intravenous injection as, for example, a sterile solution or suspension. Conventional techniques for preparing pharmaceutical compositions which can be used are described in Remington's Pharmaceutical Sciences, 1985. In short, suitable pharmaceutical preparations are made by mixing the pharmaceutical composition, preferably in purified form, with suitable adjuvants and a suitable carrier or diluent. Suitable physiological acceptable carriers or diluents include sterile water and saline. Suitable adjuvants, in this regard, include calcium, proteins (e.g. albumins), or other inert peptides (e.g. glycylglycine) or amino acids (e.g. glycine, or histidine) to stabilise the purified factor VIIa. Other physiological acceptable adjuvants are non-reducing sugars, cyclodextrins (cyclic carbohydrates derived from starch), polyalcohols (e.g. sorbitol, mannitol or glycerol), polysaccharides such as low molecular weight dextrins, detergents (e.g. polysorbate) and antioxidants (e.g. bisulfite and ascorbate). The adjuvants are generally present in a concentration of, but not limited to, from 0.001 to 4% w/v. The pharmaceutical preparation may also contain protease inhibitors, e.g. aprotinin, and preserving agents.

The preparations may be sterilized by, for example, filtration through a bacteria-retaining filter, by incorporating sterilizing agents into the compositions, by irradiating the compositions. They can also be manufactured in the form of sterile solid compositions which can be dissolved in sterile water, or some other sterile medium suitable for injection prior to or immediately before use.

The curcuminoids or derivatives thereof may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of curcuminoids. These salts can be prepared in situ during the final isolation and purification of the compounds, or by separately reacting a purified compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. “Pharmaceutical Salts”, J. Pharm. Sci. 66:1-19 (1977)).

In other cases, the compounds may contain one or more acidic functional groups and, thus, are capable of forming pharmaceutically-acceptable salts with pharmaceutically-acceptable bases. The salts can likewise be prepared in situ during the final isolation and purification of the curcuminoid containing composition, or by separately reacting derivatives comprising carboxylic or sulfonic groups with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Pharmaceutical compositions suitable for parenteral administration may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars or sodium chloride into the compositions. In addition, prolonged absorption of the injectable pharmaceutical form may be brought about by the inclusion of agents which delay absorption such as polyethylene glycol (PEG), aluminum monostearate and gelatin.

In some cases, in order to prolong the effect of a drug, it is desirable to slow the absorption of the drug from subcutaneous or intramuscular injection. This may be accomplished by coupling to PEG, the use of a liquid suspension of crystalline or amorphous material having poor water solubility. The rate of absorption of the drug then depends upon its rate of dissolution which, in turn, may depend upon size, form and amount of PEG, crystal size and crystalline form. Alternatively, delayed absorption of a parenterally-administered drug form is accomplished by dissolving or suspending the drug in an oil vehicle.

Injectable depot forms are made by forming microencapsuled matrices of the subject peptides or peptidomimetics in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions are intended for parenteral, topical or local administration for prophylactic and/or therapeutic treatment. Most preferably, the pharmaceutical compositions are administered parenterally, i.e., intravenously, so that the compositions may be rapidly transported to a selected target cell such as a cancer cell or neovascular endothelial cell. Thus, compositions are provided for parenteral administration which comprise a solution of the modified fVII molecules dissolved in an acceptable carrier, preferably an aqueous carrier. A variety of aqueous carriers may be used, e.g., water, buffered water, 0.4% saline, 0.3% glycine and the like. The modified fVIIa molecules can also be formulated into liposome preparations for delivery or targeting to sites of injury. The compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

B. Disorders to be Treated

Disorders or diseases that can be treated with the cytotoxic compound-protein conjugates include those that are characterized by the aberrant expression of tissue factor and/or angiogenesis These disorders include cancer, restenosis, and other non-malignant diseases Delivery of the compositions to the blood vessels that feed cancer cells may be useful for interrupting the supply of nutrients and oxygen to the cancer cells, thereby shrinking the tumor. Administration of the compound-protein conjugates may also be useful for treatment of metastases, which are usually inaccessible by a direct injection.

Administration of the cytotoxic-compound-protein conjugate to a target cell can modulate a physiological function of the cell by binding to a surface marker thereon and being internalized within the cell. The drug is subsequently liberated from the protein-surface marker complex and can modulate the physiological function of the target cell. In a preferred embodiment, the physiological function is proliferation of the cell, and wherein proliferation is reduced.

The target cell can be s vascular endothelial cell (VEC), a vascular smooth muscle cell (VSMC), a tumor cell, a monocyte, a macrophage and a microparticle. In a preferred embodiment, the cell is a cancer cell. In another preferred embodiment, the target cell is a vascular endothelial cell or a vascular smooth muscle cell. In other embodiments, the vascular endothelial cell can be an isolated vascular endothelial cell, a capillary endothelial cell, a venal endothelial cell, an arterial endothelial cell and a neovascular endothelial cell of a tumor. In still another embodiment, the target cell is a cultured cell.

The compounds may also be coupled to fVIIa so as to inactivate the active site of fVIIa, thereby blocking the procoagulating activity of the novel therapeutic composition. Besides acting as anticancer agents, the curcuminoid-conjugated inactivated fVIIa may also inhibit blood clotting by competing with native fVIIa. This could be a therapeutic advantage for cancer patients since many such patients experience blood clotting problems due to cancer cells that express tissue factor escaping into the circulation and triggering blood coagulation.

Other non-malignant diseases or conditions that may be treated include hypercoagulapathy, diabetic retinopathy, rheumatoid arthritis, atherosclerosis, vasculitis and skin disorder inflammation.

Diabetic retinopathy involves the uncontrollable growth of blood vessels, which express tissue factor, in the retina and leads to blindness in diabetic patients. Brain infarction results from blood clots triggered by atherosclerosis and vasculitis where tissue factor is likely to be expressed. Blood vessels of early lesions of rheumatoid arthritis also express tissue factor.

Cancer

The compounds may be used for the treatment of a number of different types of cancer. Treatment of cancer is taken to mean the treatment of primary tumors, tumor-angiogenesis, and metastases. The types of cancer that may be treated include lung cancer, cancer of head and neck, esophagus, stomach, large intestine, brain, small intestine, rectum, anus, gall bladder, kidney, bladder, liver, ureter, penis, vulva, breast, cervix, colon, prostate, ovaries; hematologic malignancies including leukemia and lymphoma; and malignant skin diseases including angiosarcoma, hemangioendothelioma, basal cell carcinoma, squamous cell carcinoma, malignant melanoma and Karposi's sarcoma.

Preferred types of cancer to be treated include leukemia, breast cancer, liver cancer, melanoma, prostate cancer, and adenocarcinoma of the lung.

Restenosis

Recent advances in the treatment of coronary vascular disease include the use of mechanical interventions to either remove or displace offending plaque material in order to re-establish adequate blood flow through the coronary arteries. Despite the use of multiple forms of mechanical interventions, including balloon angioplasty, reduction atherectomy, placement of vascular stents, laser therapy, or rotoblator, the effectiveness of these techniques remains limited by an approximately 40% restenosis rate within 6 months after treatment.

Restenosis is thought to result from a complex interaction of biological processes including platelet deposition and thrombus formation, release of chemotactic and mitogenic factors, and the migration and proliferation of vascular smooth muscle cells into the intima of the dilated arterial segment. The inhibition of platelet accumulation at sites of mechanical injury can limit the rate of restenosis in human subjects. Inhibition of platelet accumulation at the site of mechanical injury in human coronary arteries is beneficial for the ultimate healing response that occurs. While platelet accumulation occurs at sites of acute vascular injuries, the generation of thrombin at these sites may be responsible for the activation of the platelets and their subsequent accumulation.

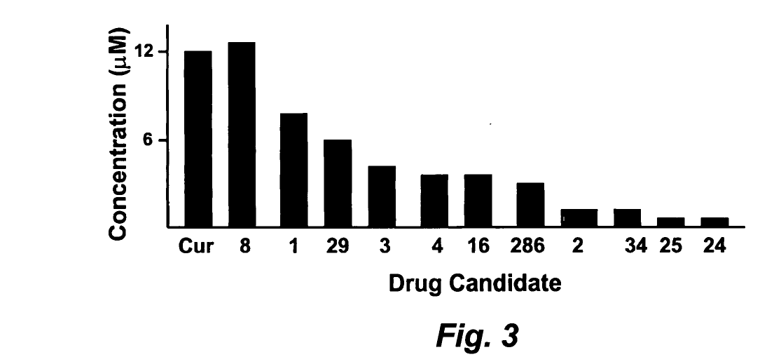

The cytotoxic compound-inactivated fVIIa conjugates may also be useful for inhibiting restenosis since they may block thrombin generation and limit platelet deposition at sites of acute vascular injury. The compositions may further inhibit restenosis by inhibiting the proliferation of endothelial or smooth muscle cells, due to internalization of the cytotoxic compounds into the cells. FIG. 3 demonstrates the cytotoxic effects of various candidate curcuminoids on endothelial cells immortalized with the Ras gene.

C. Dosages

The regimen for any patient to be treated with pharmaceutical compositions mentioned herein should be determined by those skilled in the art. The daily dose to be administered in therapy can be determined by a physician and will depend on the particular compound employed, on the route of administration and on the weight and the condition of the patient.

The pharmaceutical compositions may be administered in a single dose, but may also be given in multiple doses with intervals between successive doses depending on the dose given and the condition of the patient.

The pharmaceutical compositions may be administered intravenously or may be administered by continuous or pulsatile infusion, preferably administered by intraveneous injections.

For the treatment of skin disorders, the compositions are preferably administered systemically. For treatment of certain disorders, however, the curcuminoid-tether-linker-fVIIa may be applied topically in diseases or pathologic conditions of the skin, or locally in other tissues, to treat cancer, pre-malignant conditions and other diseases and conditions in which angiogenesis occurs.

The administration of these agents topically or locally may also used to prevent initiation or progression of such diseases and conditions. For example, a curcuminoid formulation may be administered topically or by instillation into a bladder if a biopsy indicated a pre-cancerous condition or into the cervix if a Pap smear was abnormal or suspicious.

The compositions are administered as required to alleviate the symptoms of the disorder. Assays can be performed to determine an effective amount of the compositions, either in vitro or in vivo. Representative assays are described in the examples provided below. For example, the cytotoxic effects of the curcumin-FFRck-fVIIa constructs were tested on human prostate cancer cells (Example 5), breast cancer (Example 6) and melanoma cells (Example 7), umbilical cord vascular endothelial cells (HUVECs) (Example 8) and murine vascular endothelial cells immortalized by transfection of SV40 large T antigen (MS-1 Cells). MS-1 cells are regarded as benign because the cells, when in nude mice, remain as small tumors a few millimeters in diameter during the entire life span of the mice, and do not metastasize. Normal HUVEC cells induced to express high-levels of tissue factor by exposure to phorbol ester are susceptible to the cytotoxic effect of the EF24-FFR-ck-fVIIa conjugate, as shown in Example 9.

Other methods are known to those skilled in the art, and can be used to determine an effective dose of these and other agents for the treatment and prevention of diseases or other disorders as described herein.

Suitable formulations may include those that may be administered parenterally. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the curcuminoid derivatives thereof which produces a therapeutic effect. Generally, out of one hundred percent, this amount will range from about 0.1 percent to about 99.5 percent of active ingredient, preferably from about 5 percent to about 70 percent, most preferably from about 10 percent to about 30 percent.

Methods of preparing these formulations or compositions include the step of bringing into association a compound with the carrier and, optionally, one or more accessory ingredients. In general, the formulations are prepared by uniformly and intimately bringing into association a curcuminoid-linker-fVIIa conjugate with liquid carriers, or finely divided solid carriers, or both, and then, if necessary, shaping the product.

The concentration of modified factor VII in parenteral formulations can vary widely, i.e., from less than about 0.5%, usually at or at least about 1% to as much as 15 or 20% by weight and will be selected primarily by fluid volumes, viscosities, etc., in accordance with the particular mode of administration selected.

Thus, a desirable exemplary pharmaceutical composition for intravenous infusion could be made up to contain 0.05-5 mg/kg body weight (in rats) or 0.05-10 mg/kg human adult in 250 ml of sterile Ringer's solution, and 10 mg of modified factor VII. Actual methods for preparing compositions suitable for parenteral administration will be known or apparent to those skilled in the art and are described in more detail in for example, Remington's Pharmaceutical Science, 16th ed., Mack Publishing Company, Easton, Pa. (1982), which is incorporated herein by reference.

The compositions and methods are useful in preventing or inhibiting restenosis following intervention, typically mechanical intervention, to either remove or displace offending plaque material in the treatment of coronary or peripheral vascular disease, such as in conjunction with and/or following balloon angioplasty, reductive atherectomy, placement of vascular stents, laser therapy, rotoblation, and the like. The compounds will typically be administered within about 24 hours prior to performing the intervention, and for as much as 7 days or more thereafter. Administration can be by a variety of routes as further described herein. The preferred route will be direct delivery to a blood vessel, possibly close to the site of restenosis or tissue damage for rapid and specific delivery to tissue factor-bearing cells. The compounds can also be administered systemically or locally for the placement of vascular grafts (e.g., by coating synthetic or modified natural arterial vascular grafts), at sites of anastomosis, surgical endarterectomy (typically carotid artery endarterectomy), bypass grafts, and the like. The modified fVIIa also finds use in inhibiting intimal hyperplasia, accelerated atherosclerosis and veno-occlusive disease associated with organ transplantation, e.g., following bone marrow transplantation.

The dose of modified fVII for prevention of deep vein thrombosis is in the range of about 50 μg to 500 mg/day, more typically 1 mg to 200 μg/day, and more preferably 10 to about 175 μg/day for a 70 kg patient, and administration begin at least about 6 hours prior to surgery and continue at least until the patient becomes ambulatory. The dose of the curcuminoid-fVIIa conjugates in the treatment for restenosis will vary with each patient but will generally be in the range of those suggested above.

Although preferred embodiments have been described using specific terms, devices, and methods, such description is for illustrative purposes only. The words used are words of description rather than of limitation. It is to be understood that changes and variations may be made by those of ordinary skill in the art without departing from the spirit or the scope, which is set forth in the appended claims. In addition, it should be understood that aspects of the various embodiments may be interchanged both in whole or in part.

The present invention is further illustrated by the following examples, which are provided by way of illustration and should not be construed as limiting. The contents of all references, published patents, and patents cited throughout the present application are also hereby incorporated by reference in their entireties.

### EXAMPLES

### Example 1

### Synthesis of Curcumin Analogs and Coupling of EF24 and FFRck Using a Succinate Tether

Synthesis of the conjugate of fVIIa protein and the drug molecule, EF24-FFRck-fVIIa proceeded in three steps. First, an appropriate derivative of EF24 was developed that permitted attachment to the FFR tripeptide. To synthesize EF24, piperidone.hydrate.HCl (2.2 g, 14 mmol) was suspended in glacial CH₃COOH (60 ml). The suspension was saturated with HCl gas until the solution became clear, and then treated with solid 2-fluorobenzaldehyde (5.0 g, 40 mmol). The reaction mixture was stirred at ambient temperature for 48 hrs. The precipitated solid was collected by filtration, washed with cold absolute ethanol, and dried in vacuo to give a bright-yellow crystalline solid (EF24, 4.27 g, 80% yield).

Of several compounds examined, the succinic acid derivative aa (86% yield) was suitable since it retained 50% of the activity of EF24 in cell cytotoxicity assays. To synthesize the succinyl derivative amino acid of EF24, to a solution of EF24 (0.16 g, 0.5 mmol) in anhydrous CH₂C₁₂ (6 ml) was added succinic anhydride (0.057 g, 0.5 mmol) and Et₃N (101 mg, 1 mmol). The mixture was stirred at room temperature for 3 hrs, diluted with CH₂C₁₂, washed twice with saturated NaHCO₃ (2×10 ml) and brine, dried over anhydrous Na₂SO₄, and relieved of solvent by evaporation. The resulting solid was purified by flash chromatography using benzene/acetone/acetic acid (27:10:0.5) as the eluant to obtain the yellow 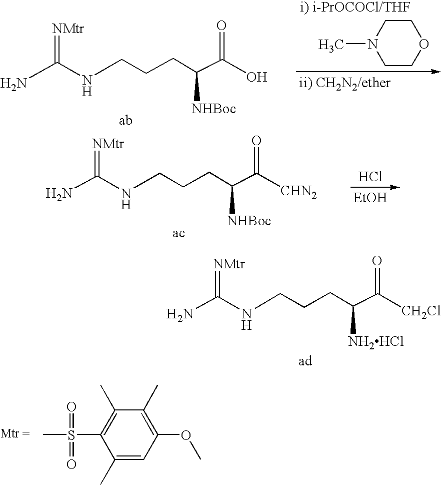
solid aa (852 mg, mp 145° C., 86% yield).

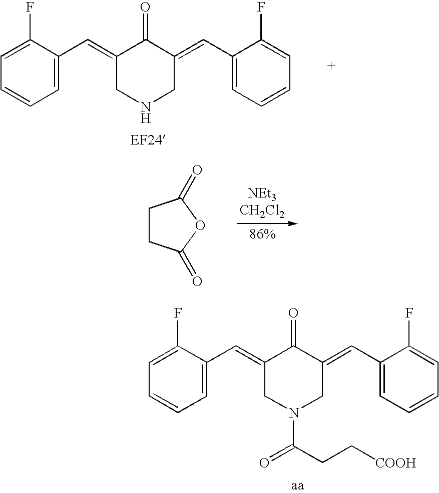

Second, the FFR-ck peptide linker was assembled as shown below. For this step, commercially available Boc-Arg(Mtr)-OH (ab 122 mg, 0.25 mmol) was dissolved in THF (2 ml) and allowed to react with isopropyl chloroformate (1.0 M in toluene, 0.25 ml, 0.25 mmol) in the presence of N-methylmorpholine (25 mg, 0.25 mmol) for 4 hrs at −20° C. The mixture was filtered, and the filtrate was added to 4 ml of ethereal diazomethane. After stirring the reaction solution for 1 hr at 0° C., the solvent was evaporated to obtain the crude product as white needles. These were purified by chromatography using ethyl acetate as the eluant to obtain a white solid, ac (75 mg, 59% yield).

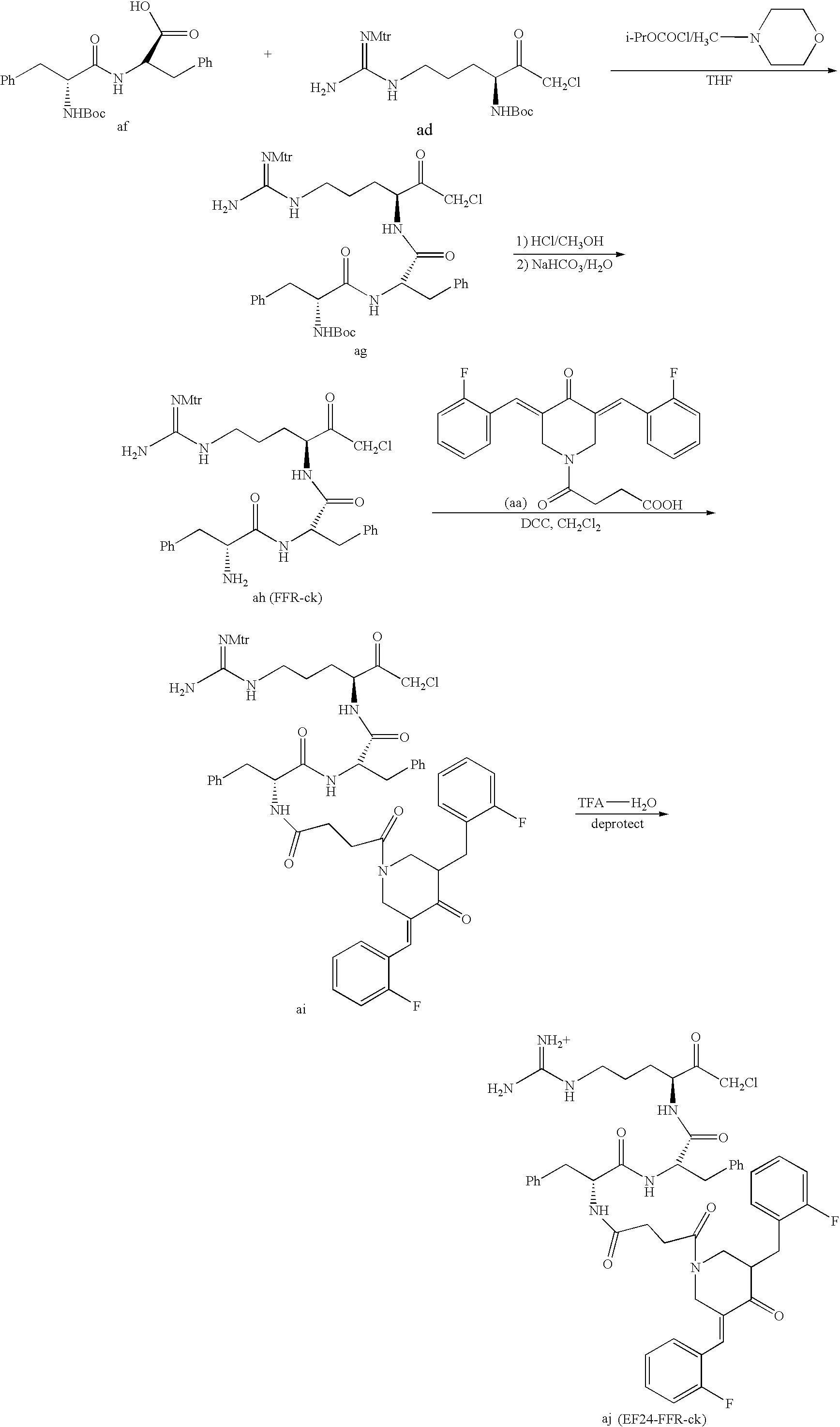

N-Boc-Phe-Phe-OH af (197 mg, 0.4 mmol) was allowed to react with N-methylmorpholine (40 mg, 0.4 mmol) and isopropyl chloroformate (1.0 M in toluene, 0.4 ml, 0.4 mmol) for 10 mins at −20° C. Cold THF (5.72 ml) containing N-methylmorpholine (40 mg, 0.4 mmol) was added to the mixture which was immediately added to Arg(Mtr)CH2Cl.HCl ad (200 mg, 0.4 mmol) dissolved in DMF (0.92 ml). After stirring for 1 hr at −20° C. and 2 hrs at room temperature, THF (5.6 ml) was added and the mixture was filtered. The filtrate was evaporated and the solid residue purified by column using EtOAc/hexanes (4:1) as the eluant. A white solid was obtained, ag (245 mg, 75% yield). Compound ag (0.05 mmol, 42.5 mg) was dissolved in EtOAc (0.16 ml) and allowed to react with methanolic HCl (0.85 mmol) at room temperature for 3.5 hrs, washed with NaHCO₃ (aq), extracted with CH₂C₁₂ (2×10 ml) and dried over MgSO₄ and filtered. Evaporation of the solvent furnished a white solid, FFR-ck ah (40 mg).

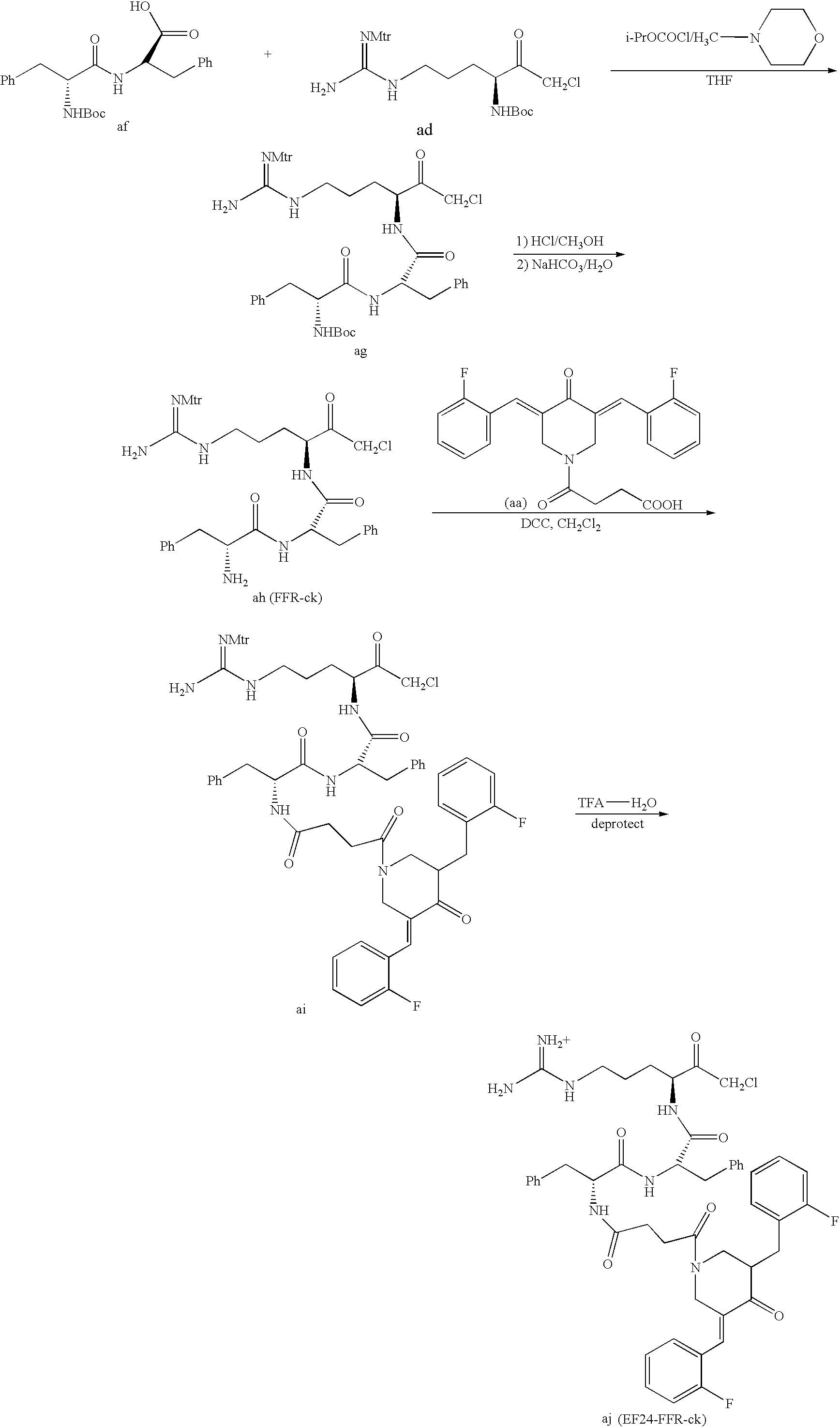

To a mixture of ah (24 mg, 0.032 mmol) and aa (12 mg, 0.03 mmol) in CH₂C₁₂ (0.6 ml) was added DCC (6.18 mg, 0.03 mmol). After stirring overnight at room temperature, filtration and evaporation of the solvent, and purification by flash chromatography using ethyl acetate as the eluant ai (17 mg, 49% yield) was obtained. Compound ai (34 mg, 0.03 mmol) was dissolved in 95% aqueous TFA (0.95 ml) with thioanisole (0.05 ml). The resulting dark solution was stirred for 48 hrs at room temperature and then concentrated under vacuum. The resulting solid was triturated with ether, recrystallized and dried under a vacuum to supply compound aj (EF24-FFR-ck) (12 mg, 45% yield).

### Example 2

### Coupling of EF24-FFRck (aj) and fVIIa

Method 1: Recombinant fVIIa (250 μg) was resuspended in 0.5 ml of distilled water and dialyzed in 1 liter of 1 mM TrisHCI, pH 8.0 at 4° C. overnight. Forty-fold molar excess of EF24-FFRck aj synthesized as described in Example 2 above, in 0.25 ml of DMSO was added to a final concentration of 400 μM. The mixture was covered with aluminum foil (EF24 is photosensitive) and incubated at room temperature overnight in darkness. The reaction mixture was centrifuged at 16,000 rpm at 4° C. for 20 minutes in a Sorvall centrifuge to precipitate unbound EF24-FFRck and separate it from EF24-FFRck-fVIIa. The supernatant was further dialyzed in 100 ml of sterile cell culture medium containing 10% fetal bovine serum, penicillin (100 units/ml) and streptomycin 100 μg/ml) at 4° C. overnight. EF24-FFRck-fVIIa equilibrated with the culture medium was added to cells in wells of a 96-well plate.

Method 2: (1). Factor VIIa (fVIIa) is dialyzed against 1.0 mM Tris HCl, pH 7.4 at 4° C. overnight. (2) EF24-FFRck is dissolved in 100% DMSO. (3) fVIIa per ml and EF24-FFRck per 0.25 ml is mixed at a molar ratio 1:13.2 and gently stirred for 2 hrs at room temperature. (4) Additional EF24-FFRck per 0.25 ml (at a molar ratio of 1:13.2) was added to the reaction mixture to make the final molar ratio 1:40 and continue the coupling reaction overnight at room temperature. (5) The coupled product EF24-FER-ck-fVIIa was separated from uncoupled EF24-FFRck by column chromatography and 0.5 ml fractions collected. (6) a protein peak (fVIIa) was determined by reading fractions at OD₂₈₀ and the Bradford protein determination (Bio-Rad). (7) Active fractions were pooled.

The resulting coupled EF24-FFRck (al) to fVIIa was analyzed by mass spectroscopy.

Mass for fVIIa_is 52392.6+H Daltons, and for EF24-FFRck-fVIIa is 54322.2+H Daltons. The mass of the latter is 1929.6 Dalton greater than the former. MW of EF24-FFRck(894)−HCI(37)=857. 1929.6 divided by 857=2.3. At least 2 molecules of EF24-FFRck were covalently attached to fVIIa.

### Example 3

### F24-FFRck-fVIIa Binds Only TF via fVIIa and Kills Human Prostate Cancer Cell Lines

Tissue factor (TF) and vascular endothelial growth factor (VEGF) levels expressed by DU145 and PC3 prostate cancer cell lines were measured by ELISA, as shown in Table 1 below. High TF and VEGF levels were found in DU145 cells.

TABLE 1
Tissue factor (TF) and vascular endothelial growth factor (VEGF)
ELISA in DU145 and PC3 prostate cancer cell lines.
High TF and VEGF levels
in DU145 cells. Values indicate Mean ± S.D.

| | |
|---|---|
| TF (pg/ml) | VEGF (pg/ml) |

| DU-145 | PC-3 | DU-145 | PC-3 |
|---|---|---|---|
| 10690 ± 650 | 230 ± 16 | 30511 ± 5748 | 2186 ± 307 |

DU145 cells were plated with 2×10⁴ cells/100 μl/well in a 96 well plate and cultured overnight. The cells were cultured for 48 hrs. Cultures was terminated by adding 40% TCA to a final concentration of 10%. Cells were fixed in TCA at 4° for 1 hr, washed with tap water 5 times and air dried. Sulforhodamine B (SRB) solution (100 μl) at 0.4% (w/v) in 1% acetic acid was added to each well, and the cells were incubated for 10 mins at room temperature. After staining, unbound dye was removed by washing five times with 1% acetic acid and air dried. Bound dye was subsequently solubilized with 200 μl of 10 mM Trizma base, and the absorbance was read on an automated plate reader at a wavelength of 490 nm. Assays were performed in triplicate or quadruplicate. An asterisk (*) indicates p<0.0001 by the Student t-test (two-tailed probability). The concentration of EF24-FFRck-fVIIa was estimated based on protein concentration.

EF24-FFRck alone does not kill any cells since it cannot bind the cell surface, as shown in Table 2.

TABLE 2
EF24-FFRck-fVIIa kills DU145, a Human Prostate Cancer Cell
Line which expresses Tissue Factor. SRB Viability Test.
Values are Mean S.D.

| | O.D.570 nm |
|---|---|
| Control (0.5% DMSO) | 0.370 ± 0.015 |
| EF24-FFRck-fVIIa, 0.8 μM | 0.333 ± 0.053 |
| EF24-FFRck-fVIIa, 8 μM | 0.111 ± 0.004* |
| EF24, 0.8 μM | 0.391 ± 0.041 |
| EF24, 8 μM | 0.053 ± 0.025* |
| EF24-FFRck, 0.8 μM | 0.389 ± 0.021 |
| EF24-FFRck, 8 μM | 0.383 ± 0.027 |

### Example 4

### EF24-FFRck-fVIIa Kills Human Breast Cancer and Melanoma Cells

The Neutral Red (NR) dye viability assay, instead of the Sulforhodamine B (SRB) assay, was used. In the NR viability assay, NR dye is taken up only by viable cells, while in the SRB viability assay, viable cells are fixed by trichloracetic acid (TCA) on the plate (thus, cells are killed), and the fixed cells are stained by SRB dye.

At the termination of culture, medium was removed and 200 μl of fresh, warm medium containing 50 μg of NR/ml was added to each well in a 96-well plate. Cells were incubated at 37° for 30 mins, followed by two washes with 200 μl of PBS. The NR taken up by cells was dissolved by adding 200 μl of 0.5N HCI containing 35% ethanol. The amount of the dye in each well was read at 570 nm by an ELISA plate reader. The results are shown in Table 3.

TABLE 3
EF24-FFRck-fVIIa kills Human Breast Cancer (MDA-MB-231)
and Melanoma (RPMI-7951) NR Viability Test.
Values indicate Mean ± S.E.

| | O.D.570 nm | |
|---|---|---|
| | MDA231 | RPM17951 |
| Control (0.5% DMSO) | 0.193 ± 0.019 | 0.269 ± 0.019 |
| EF24-FFRck-fVIIa, 0.5 pM | 0.142 ± 0.010 | 0.292 ± 0.028 |
| EF24-FFRck-fVIIa, 2 pM | 0.041 ± 0.002* | 0.066 ± 0.002* |
| EF24, 1 pM | 0.172 ± 0.020 | 0.220 ± 0.023 |
| EF24, 2 pM | 0.109 ± 0.014* | 0.119 ± 0.018 |
| EF24-FFRck, 1 pM | 0.191 ± 0.013 | 0.253 ± 0.018 |
| EF24-FFRck, 2 pM | 0.171 ± 0.009 | 0.247 ± 0.020 |

Student t-test (two-tailed probability)(95% confident level)

### Example 5

### EF24-FFRck-fVIIa Has No Effect on Normal Human Melanocytes and Normal Human Breast Luminal Ductal Cells

The effect of EF24-FFRck-fVIIa on normal human melanocytes and normal human breast luminal ductal cells.

The results shown in Table 4 demonstrate the non-toxicity of the conjugate for normal cells.

TABLE 4
EF24-FFRck-fVIIa has no effect on normal human melanocytes
and MCF10 (normal human breast luminal ductal cell line) which do not
express Tissue Factor: NR (neutral red dye) Viability Test. Values are
Mean ± S.D.

| | O.D 570 nm | |
|---|---|---|
| | Melanocytes | Normal Breast Cells |
| Control (None) | 0.264 ± 0.023 | 0.106 ± 0.006 |
| DMSO (1%) | 0.261 ± 0.012 | 0.107 ± 0.012 |
| EF24-FFRck-fVIIa, 4 μM | 0.210 ± 0.005 | 0.096 ± 0.023 |
| EF24, 0.8 μM | 0.255 ± 0.009 | 0.104 ± 0.018 |
| EF24, 4 μM | 0.119 ± 0.009* | 0.091 ± 0.007** |
| EF24-FFRck, 0.8 μM | 0.252 ± 0.007 | 0.101 ± 0.013 |
| EF24-FFRck, 4 μM | 0.249 ± 0.015 | 0.113 ± 0.003 |

*p = 0.002,
**p = 0.031

Assays were performed essentially the same as for DU145 above.

### Example 6

### EF24-FFRck-fVIIa Does Not Kill Normal HUVECs

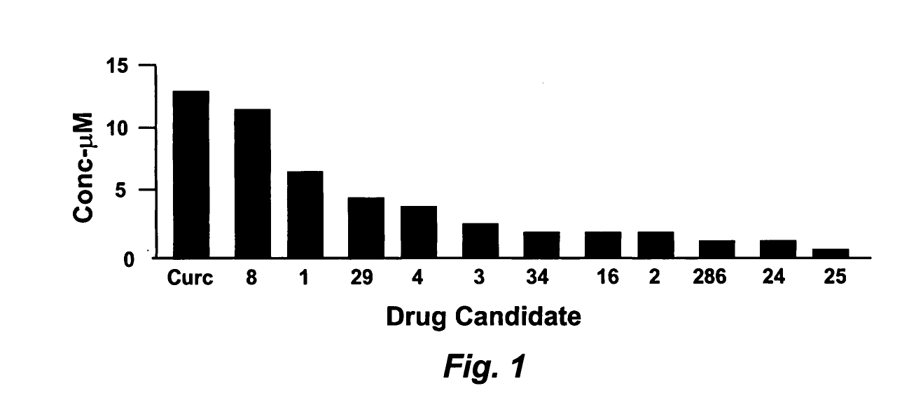

As demonstrated by FIG. 1 and Table 5, EF24-FFRck-fVIIa does not kill normal HUVECs that do not express surface bound tissue factor.

TABLE 5
EF24-FFRck-fVIIa does not kill normal HUVECs that do
not express tissue factor: SRB Viability Test (NCI method).
Mean ± S.D.

| | O.D. 490 nm |
|---|---|
| Control (0.5% DMSO) | 0.119 ± 0.003 |
| EF24-FFRck-fVIIa, 0.8 μM | not done |
| EF24-FFRck-fVIIa, 8 μM | 0.370 ± 0.027^{a} |
| EF24, 0.8 μM | 0.136 ± 0.010 |
| EF24, 8 μM | 0.038 ± 0.010* |
| EF24-FFRck, 0.8 μM | 0.152 ± 0.026 |
| EF24-FFRck, 8 μM | 0.160 ± 0.038 |

*Student t-test (two-tailed probability)(95% confident level)
^{a}Cells were not washed before adding the SRB dye and therefore precipitated EF24-FFRck-fVIIa adsorbed dye thereby giving a false elevated .D. 490 nm value

### Example 7

### EF24-FFRck-fVIIa Kills HUVECs Induced to Express TF by 100 nM TPA

As shown by Table 6, EF24-FFRck-fVIIa in a dosage of 0.6 micromolar, kills HUVECs induced to express TF by 100 nM TPA.

TABLE 6
EF24-FFRck-fVIIa kills HUVECs induced to express TF by 100 nM
TPA (phorbol ester) for 24 hrs prior to adding EF24-FFRck-fVIIa: SRB
Viability Test (NCI method). Mean ± S.D.

| | TPA | O.D. 490 nm |
|---|---|---|
| EF24-FFRck-fVIIa, 0.6 μM | 0 | 0.170 ± 0.015 |
| EF24-FFRck-fVIIa, 0.6 μM | + | 0.059 ± 0.004* |

*Student t-test (two-tailed probability)(95% confident level)

### Example 8

### Curcumin Analogs (A279L. A279U and EF-15) Are Not Cytotoxic to Vascular Endothelial Cells

HUVECs, MS-1 cells and SVR cells were cultured to confluence and agents were incubated for 24 hrs with curcumin analogs. Cell viability was determined by Neutral Red assays.

Curcumin and mono-carbonyl derivatives of curcumin, diarlylpentanoids, were tested for their anti-proliferative effects against a murine endothelial cell line transformed with mutated H-ras (FIG. 3) and a breast/melanoma cell line.

Curcumindiferuloylmethane, 1,7-bis-(4-hydroxy-3-methoxyphenyl)-1,6-heptadiene-3,5-dione), (1,5-Bis-(4-hydroxyphenyl)-penta-1,4-dien-3-one (1), 1,5-Bis-(2-hydroxyphenyl)-penta-1,4-dien-3-one (2), 1,5-Bis-(3-hydroxyphenyl)-penta-1,4-dien-3-one (3), 2,6-Bis-(2-hydroxybenzylidene)-cyclohexanone (4), 3,5-Bis-(2-hydroxybenzylidene)-tetrahydro-4-H-pyran-4-one (25), 3,5-Bis-(2-hydroxybenzylidene)-1-methyl-4-piperidone (34), 1,5-Bis-(2-fluorophenyl)-penta-1,4-dien-3-one (8), 3,5-Bis-(2-fluorobenzylidene)-tetrahydro-4-H-pyran-4-one (29), 1,5-Bis-(2-methoxyphenyl)-penta-1,4-dien-3-one (16), 1,5-Bis-(2-acetylphenyl)-penta-1,4-diene-3-one (286): 3,5-Bis-(2-fluorobenzylidene)-piperidin-4-one, acetic acid salt (24).

The results are shown in Table 7. Among synthetic curcumin analogs, A279L, A279U and EF-15 were not cytotoxic at 20 μM. MS-I cells were murine vascular endothelial cells which were immortalized by transfection of SV40 large T antigen but are non-maligant. However, when MS-1 cells were transfected with a ras mutant gene, cells were transfromed to become malignant angiosarcoma cells, (SVR cells).

TABLE 7
Curcumin analogs (A279L. A279U and EF-15) are not cytotoxic to
vascular endothelial cells.

| | | Neutral Red Viability | | |
|---|---|---|---|---|
| | | Assay (% of Control) | | |
| | | HUVECs | MS-1 Cells | SVR cells |
| DMSO (0.1%) | (control) | 100 | 100 | 100 |
| Curcumin | (1 μM) | 103 | 90 | 88 |
| | (20 μM) | 21 | 3 | 4 |
| A279L | (20 μM) | 97 | 90 | 90 |
| A279U | (20 μM) | 92 | 96 | 92 |
| EF-15 | (20 μM) | 100 | 95 | 100 |
| C.V.6 | (1 μM) | 60 | 33 | 62 |
| | (10 μM) | 26 | 10 | 3 |
| C.V.10 | (1 μM) | 100 | 68 | 75 |
| | (10 μM) | 17 | 7 | 5 |
| EF-2 | (1 μM) | 24 | 8 | 20 |
| | (10 μM) | 9 | 3 | 2 |
| EF-4 | (1 μM) | 14 | 4 | 7 |
| | (10 μM) | 15 | 4 | 6 |
| EF-17 | (1 μM) | 93 | 87 | 75 |
| | (10 μM) | 47 | 10 | 12 |
| EF-25 | (1 μM) | 17 | 6 | 28 |
| | (10 μM) | 17 | 6 | 4 |
| A283 | (1 μM) | 38 | 21 | 37 |
| | (10 μM) | 17 | 4 | 3 |
| A286 | (1 μM) | 30 | 11 | 24 |
| | (10 μM) | 17 | 8 | 4 |
| A287 | (1 μM) | 80 | 43 | 67 |
| | (10 μM) | 15 | 6 | 4 |

### Example 9

### Internalization of TF/FFR-ck-VIIa Complexes After Incubating Cells with Varying Concentrations of FFR-ck-fVIIa for 24 hrs

In three human cancer cell lines (high TF and VEGF producers), FFR-ck-VIIa alone caused internalization of TF into caveolae in the plasmalemma vesicles (Triton X-100 insoluble region of cell membrane) in a dose-dependent manner. FFR-ck-VIIa totally inhibited TF, which remained on the cell surface, to catalyze factor X to generate factor Xa. However, VEGF production and cell viability were not affected. In MDA-MB-231 cells, approximately 10 μM of FFR-ck-VIIa will be required to internalize 50% of TF-FFR-ck-VIIa complexes because MDA-MB-231 human breast cancer cells express greater level of TF than other cell lines.

TABLE 8
Effect of FFRck-fVIIa on cancer cells.

| Tumor Cell Line | 0 | 100 | 1000 |
|---|---|---|---|
| | TF (nM) on the cell surface | | |
| | FFR-ck-VIIa (nM) | | |
| Hs294T | 6.0 + 0.7 | 3.9 + 0.6* | 2.5 + 0.3* |
| RPMI7951 | 81.6 + 4.5 | 38.7 + 1.4* | 35.0 + 6.2* |
| MDA-MB-231 | 624.8 + 42.0 | 465.5 + 17.7* | 488.9 + 1.6* |
| | Percentage relative to 0 nM | | |
| | FFR-ck-VIIa control | | |
| Hs294T | 100 | 65* | 42* |
| RPMI7951 | 100 | 76* | 48* |
| MDA-MB-231 | 100 | 91* | 80* |
| | Percentage internalized relative to | | |
| | 0 nM FFR-ck-VIIa control | | |
| Hs294T | 0 | 35 | 58 |
| RPMI7951 | 0 | 24 | 52 |
| MDA-MB-231 | 0 | 9 | 20 |

Values of TF indicate mean + S.D. of triplicate determinations.
*Statistically significantly different from control values (p < 0.05).

### Example 10

### Curcuminoid EF24 is More Effective than Curcumin Against Tumor Cells

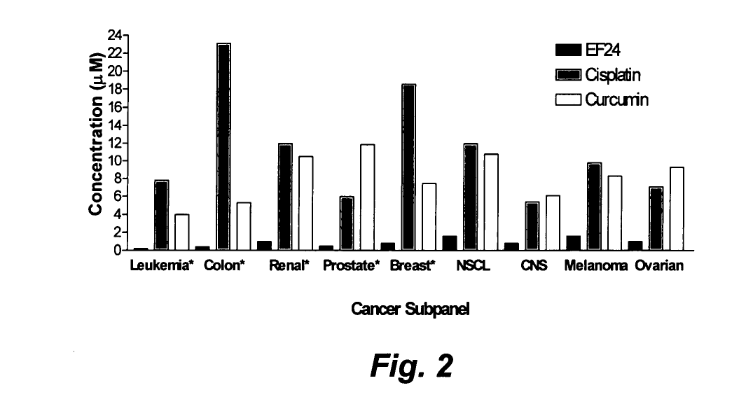

Curcumin, EF24 and cisplatin were tested in vitro against a panel of 60 cancer cell lines in the NCI screening system. The results demonstrated that one of the curcuminoids, 3,5-Bis-(2-fluorobenzylidene)-piperidin-4-one, acetic acid salt (EF24) was significantly more effective than either cisplatin or curcumin, as shown in FIG. 2. Curcumin and mono-carbonyl derivatives of curcumin, diarlylpentanoids, were also added to transformed breast cancer cells and the mean growth inhibitory concentrations determined, as shown in FIG. 3. The curcuminoids that were added include curcumin (diferuloylmethane, 1,7-bis-(4-hydroxy-3-methoxyphenyl)-1,6-heptadiene-3,5-dione); (1,5-Bis-(4-hydroxyphenyl)-penta-1,4-dien-3-one (1); 1,5-Bis-(2-hydroxyphenyl)-penta-1,4-dien-3-one (2); 1,5-Bis-(3-hydroxyphenyl)-penta-1,4-dien-3-one (3); 2,6-Bis-(2-hydroxybenzylidene)-cyclohexanone (4); 1,5-Bis-(2-fluorophenyl)-penta-1,4-dien-3-one (8); 1,5-Bis-(2-methoxyphenyl)-penta-1,4-dien-3-one (16); 3,5-Bis-(2-fluorobenzylidene)-piperidin-4-one, acetic acid salt (24); 3,5-Bis-(2-hydroxybenzylidene)-tetrahydro-4-H-pyran-4-one (25); 5-Bis-(2-fluorobenzylidene)-tetrahydro-4-H-pyran-4-one (29); 3,5-Bis-(2-hydroxybenzylidene)-1-methyl-4-piperidone (34); 1,5-Bis-(2-acetylphenyl)-penta-1,4-diene-3-one (286).

### Example 11

### Methods of Conjugating Factor VIIa and EF24-FFRck Without Precipitation

A fVIIa solution (M.W. 50,000) was dialyzed in a 1000 times greater volume of 1 mM Tris-Cl, pH 7.5, containing 0.1% Tween 80 for four days, changing the buffer once every 12 hours until no precipitation is seen. The dialysis membrane had a M.W. cut-off in the range 10,000-13,000. The resulting FVIIa solution should be clear before the conjugation step.

Sufficient EF24-FFRck (M.W. 894) was weighed out to provide a molar ratio of EF24-FFRck to fVIIa at 20 to 1.

EF24-FFRck was dissolved in 100% DMSO. The volume of DMSO is approximately the same volume of the fVIIa solution.

While gently stirring fVIIa at 4° C. in a cold room, aliquots of ⅕ of the volume of EF24-FFRck in DMSO were added hourly until all of the solution was added. This is done because a half-life of EF24-FFRck in an aqueous solution may be a few hours. There should be no precipitation or cloudiness observed during the conjugation reaction. The reaction should be carried out in darkness due to photosensitivity of EF24. The reaction was continued overnight.

The next morning, the reaction mixture was dialyzed for 24 hours in a 1000 times greater volume of 1 mM Tris-Cl, pH 7.5 containing 0.1% Tween 80, changing the buffer after 12 hours. This process removes DMSO and unconjugated free EF24-FFRck

The solution containing the concentrate was concentrated using centricon-30 (that filters molecules smaller than M.W. 30,000) by centrifuging in a Sorvall SS-34 rotor (a mid point radius is 9 cm, while radius at the top is 6 cm and at the bottom is 12 cm) at 5,000 rpm. This concentration procedure further filters out free EF24-FFRck in addition to concentrate the conjugate solution.

RCF=1.118×10−5×radius at the centricon filter membrane (9 cm)×(rpm)²
RCF at 3000, rpm=4458
RCF at 4000, rpm=5459

The conjugate, but not the filtrate, exhibited cytotoxic activity against tissue factor expressing cancer cells.

1. A conjugate comprising:
(a) a protein selected from the group consisting of factor VII protein, antibody to tissue factor and tissue factor pathway inhibitor selectively binding tissue factor on a target cell;
(b) at least one hydrolysable tether covalently bonded to the protein; and
(c) a cytotoxic compound bonded to the tether by a hydrolysable bond,
wherein the tissue factor internalizes the conjugate when it binds to the tissue factor.

2. The composition according to claim 1, wherein the protein is factor VII or a component polypeptide of factor VIIa.

3. The composition according to claim 2, wherein the polypeptide comprises the amino acid sequence between amino acid positions 153 and 406 of SEQ ID NO: 1 or a truncated or modified variant thereof.

4. The composition according to claim 1, wherein the protein is an antibody or a tissue factor pathway inhibitor.

5. The composition according to claim 1, wherein the tether further comprises at least one linker.

6. The composition according to claim 5, wherein the at least one linker is a peptidyl methylketone linker.

7. The composition according to claim 1, wherein the hydrolysable bond is selected from the group consisting of a carbamate, an amide, an ester, a carbonate and a sulfonate.

8. The composition according to claim 5, wherein the at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

9. The composition according to claim 1, wherein the tether is covalently bonded to an amino acid side chain within a serine protease active site of factor VIIa, thereby inactivating the serine protease active site.

10. The composition according to claim 1, wherein the cytotoxic compound is a curcuminoid having the formula:
wherein: 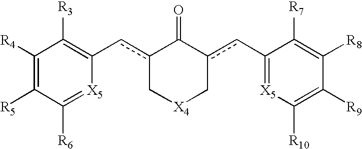
X₄ is (CH₂)m, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl;
m is 1-7;
each X₅ is independently N or C—R₁₁;
and each R₃—R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, or trialkylammonium; and the dashed lines indicate optional double bonds; with the proviso that when X₄ is (CH₂)ₘ, m is 2-6, and each X₅ is C—R₁₁, R₃—R₁₀, are not alkoxy, and when X₄ is NR₁₂ and each X₅ is N, R₃—R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

11. The composition according to claim 10, wherein X₄ is selected from the group consisting of —NH and —NR₁₂.

12. The composition according to claim 10, wherein R₃—R₁₀ is selected from hydroxyl and —NHR₁₂.

13. The composition according to claim 1, wherein the cytotoxic compound is a curcuminoid having the formula:

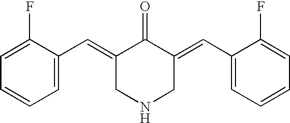

14. The composition according to claim 1, wherein the tether is selected from the group consisting of a dicarboxylic acid, a disulfonic acid, an omega-amino carboxylic acid, an omega-amino sulfonic acid, an omega-amino carboxysulfonic acid, succinate or a derivative thereof, wherein the tether comprises 2-6 carbons, and wherein the tether is capable of forming a hydrolysable bond.

15. The composition further comprising a pharmaceutically acceptable carrier.

16. A method of producing a cytotoxic compound-protein conjugate, comprising the steps of:
(a) providing a cytotoxic compound;
(b) bonding covalently the product of step (a) via a hydrolysable tether to a protein selected from the group consisting of factor VII protein, antibody to tissue factor and tissue factor pathway inhibitor selectively binding tissue factor on a target cell,
wherein the tissue factor internalizes the conjugate when it binds to the tissue factor.

17. The method of claim 16, wherein the cytotoxic compound is a curcuminoid having the formula: 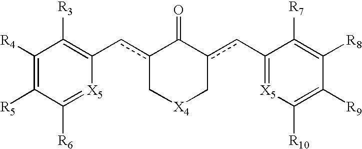
X₄ is (CH₂)ₘ, O, S, SO, SO₂, or NR₁₂, where R₁₂ is H, alkyl, substituted alkyl, acyl, alkoxycarbonyl, aminocarbonyl, alkylaminocarbonyl or dialkylaminocarbonyl;
m is 1-7;
each X₅ is independently N or C—R₁₁;
and each R₃—R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide, alkylcarbonyl, acyl, or trialkylammonium; and the dashed lines indicate optional double bonds; with the proviso that when X₄ is (CH₂)ₘ, m is 2-6, and each X₅ is C—R₁₁, R₃—R₁₁ are not alkoxy, and when X₄ is NR₁₂ and each X₅ is N, R₃—R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

18. The method of claim 17, wherein step (a) comprises reacting the curcuminoid with a tether selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

19. The method of claim 17, wherein X₄ is selected from the group consisting of —NH and —NR₁₂.

20. The method of claim 17, wherein R₃—R₁₀ is selected from hydroxyl and —NHR₁₂.

21. The method of claim 17, wherein the cytotoxic compound has the formula:

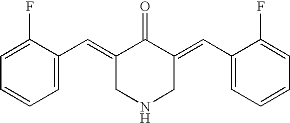

22. The method of claim 16, wherein step (a) comprises reacting the cytotoxic compound with a dicarboxylic anhydride.

23. The method of claim 22, wherein the dicarboxylic anhydride is succinic anhydride.

24. The method of claim 23, wherein the product of step (a) has the formula:

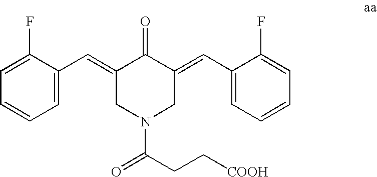

25. The method of claim 16, wherein step (b) further comprises providing a peptidyl linker attached at one end to the tether.

26. The method of claim 16, wherein the step (b) comprises the steps of:
(i) reacting a composition having the formula: 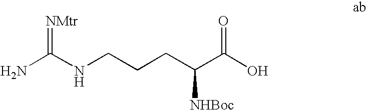
with isopropyl chloroformate and ethereal diazomethane, thereby producing a compound having the formula: 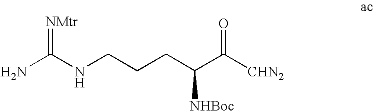
(ii) reacting a compound having the formula: 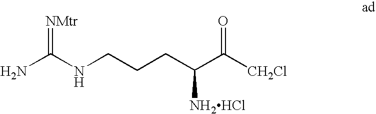
with N-Boc-Phe-Phe-OH, isopropyl chloroformate, and a base; thereby producing a compound having the formula: 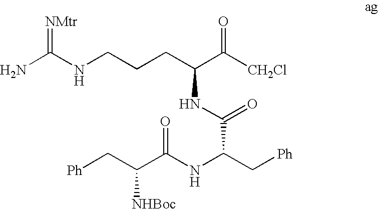
(iii) deprotecting compound ag, thereby producing a compound having the formula:

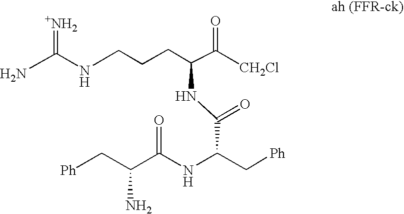

27. The method of claim 26, wherein the composition of step (b) has the formula:

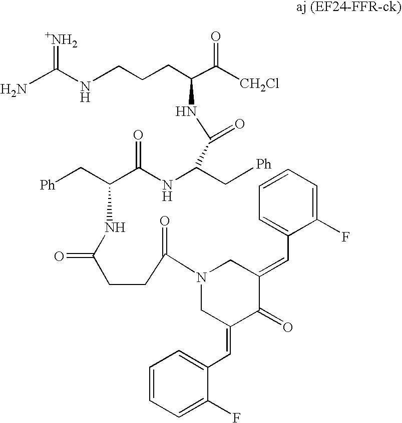

28. The method according to claim 16, further comprising the step of dialyzing a solution containing the protein prior to covalently bonding the composition of step (b) to the protein.

29. The method according to claim 16, further comprising the step of dissolving the composition of step (b) in a solvent to provide a molar ratio of the composition of step (b) to protein of greater than 1 to 1.

30. The method according to claim 29, wherein the molar ratio is 20 to 1.

31. The method according to claim 29, wherein the solvent is a nonaqueous, polar solvent selected from the group consisting of oils, alcohols, amides, esters, ethers, ketones, hydrocarbons, and mixtures thereof.

32. The method according to claim 31, wherein the solvent is dimethyl sulfoxide (DMSO).

33. The method according to claim 29, wherein the solvent is an aqueous liquid selected from the group consisting of water, saline solution, dextrose solution, and electrolyte solution.

34. The method according to claim 16 further comprising the step of adding aliquots of a fraction of the volume of the composition of step (b) to the protein.

35. The method according to claim 16, further comprising the step of dialyzing the conjugate to remove solvent.

36. A method of killing a target cell expressing tissue factor on its surface, comprising the steps of contacting the target cell with a conjugate composition comprising a protein selected from the group consisting of factor VII protein, antibody to tissue factor and tissue factor pathway inhibitor selectively binding tissue factor, wherein the conjugate is internalized and the cytotoxic compound released from the protein to kill the target cell.

37. The method according to claim 36, wherein the target cell is selected from a vascular endothelial cell, a vascular smooth muscle cell, a tumor cell, a monocyte, a macrophage and a microparticle.

38. The method according to claim 36, wherein the vascular endothelial cell is selected from the group consisting of an isolated vascular endothelial cell, a capillary endothelial cell, a venal endothelial cell, an arterial endothelial cell and a neovascular endothelial cell of a tumor.

39. The method according to claim 36, wherein the composition is delivered to an animal or human by a route selected from the group consisting of topical intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intrasternal injection and infusion.
